(19) United States
(12) Patent Application Publication
(10) Pub. No.: US 2008/0226653 A1
(43) Pub. Date: Sep. 18, 2008

(54) Inhibition of Platelet Aggregation

(75) Inventors: Ratna Bose, Winnipeg (CA); Diane E. Roberts, Winnipeg (CA)

Correspondence Address: FULBRIGHT & JAWORSKI L.L.P., 600 CONGRESS AVE., SUITE 2400, AUSTIN, TX, 78701, US

(73) Assignee: THE UNIVERSITY OF MANITOBA, Winnipeg (CA)

(21) Appl. No.: 11/875,398

(22) Filed: Oct. 19, 2007

Related U.S. Application Data
(60) Provisional application No. 60/862,275, filed on Oct. 20, 2006.

(51) Int. Cl. A61K 39/395; A61P 3/08

(52) U.S. Cl. 424/152.1; 424/172.1

(57) ABSTRACT

The present invention relates generally to the field of hyperglycemia-induced complications. More particularly, it concerns methods and compositions for the treatment and prevention of microvascular complications associated with diabetes. In one embodiment, the present invention provides a method of inhibiting hyperglycemia-induced platelet activation in a subject by administering to the subject an effective amount of a TRPC6 inhibitor.

### BACKGROUND OF THE INVENTION

This application claims the benefit of priority to U.S. Provisional Patent Application No. 60/862,275, filed Oct. 20, 2006, the entire contents of which are specifically incorporated herein.

1. Field of the Invention

The present invention relates generally to the field of hyperglycemia-induced complications. More particularly, it concerns methods and compositions for the treatment and prevention of microvascular complications associated with diabetes.

2. Description of Related Art

A major goal of therapeutic treatment of diabetic patients is to delay or prevent the complications associated with chronic hyperglycemia. Cardiovascular complications are the most frequent cause of morbidity and mortality in diabetic patients (Morrish et al., 1990). These complications include microangiopathy, retinopathy, neuropathy, nephropathy, and macroangiopathy, which is an accelerated form of atherosclerosis.

It is postulated that cardiovascular complications develop in diabetic patients because of abnormal platelet activation (Colwell et al., 1983; Pyorala et al., 1987). It has been demonstrated that platelets from both type I and type II diabetic patients (preclinical diabetes, clinical diabetes, and diabetic patients with complications) exhibit hyperactivity in vitro and in vivo. This hyperactivity includes increased platelet adhesion, aggregation, thromboxane production, increased plasma levels of platelet-specific proteins, and increased platelet turnover (Bern et al., 1978; Collwell et al., 1981; Colwell et al., 1983). In addition, in vivo thrombosis can occur more readily in large vessels in response to injury in diabetic patients (Honour et al., 1976). Platelet aggregates (microthrombi) have also been shown to exist in the small vessels of the retina in diabetic patients and animals (Dobbie et al., 1974; Ihibashi et al., 1981).

Microvascular complications are prevalent in diabetics. Classic microvascular complications of diabetes include retinopathy, nephropathy, and neuropathy. Diabetic retinopathy is marked by changes in the vasculature of the retina and is the leading cause of blindness in American adults. Diabetic nephropathy is marked by a decreased ability to properly filter blood in the kidneys such that large blood proteins, such as albumin, pass into the urine and are lost. Diabetic nephropathy is the leading cause of kidney failure in the United States. Diabetic neuropathies can affect nerves throughout the body and commonly cause numbness and pain in the hands, arms, feet and legs. Approximately 50% of diabetics suffer from some form of neuropathy, and the likelihood of developing some form of neuropathy as a complication of diabetes increases over time.

To date, treatments for microvascular complications of diabetes are limited to those that attempt to slow the progression of the complication. There are no effective treatment interventions to prevent or alleviate hyperglycemia-induced microvascular complications. Aspirin inhibits the arachidonate pathway, a common amplifying pathway for many agonists during platelet activation that is found to be upregulated in platelets from diabetics. However, in clinical trials, aspirin had only limited efficacy in preventing chronic complications in diabetic patients (Patrono et al., 1993). Therefore, there is a need for novel therapeutics that prevent and alleviate hypergylcemia-induced microvascular complications.

### SUMMARY OF THE INVENTION

The present disclosure overcomes deficiencies in the art by providing compositions and methods for the prevention and/or treatment of microvascular complications of hyperglycemia. In certain aspects, the compositions and methods described herein are used to prevent or treat microvascular complications of diabetes. In specific embodiments, the present disclosure describes the use of TRPC6 inhibitors to prevent hyperglycemia-induced platelet activation.

In certain aspects, the method of inhibiting hyperglycemia-induced platelet activation via administration of an effective amount of a TRPC6 inhibitor further comprises assessing the blood glucose level of the patient. Blood glucose level may be assessed, for example, by the Fasting Plasma Glucose Test (FPG) or the Oral Glucose Tolerance Test (OGTT). In some embodiments, a TRPC6 inhibitor is administered to a subject with an FPG of greater than 125 mg/dL. In other embodiments, a TRPC6 inhibitor is administered to a subject with an FPG of greater than 99 mg/dL. In yet other embodiments, a TRPC6 inhibitor is administered to a subject with an OGTT of greater than 199 mg/dL or greater than 139 mg/dL.

In some embodiments, the present invention provides methods for treating a diabetic complication in a subject having chronic hyperglycemia comprising administering to the subject an effective amount of a TRPC6 inhibitor. Treating a diabetic complication may include alleviating, delaying the onset, or preventing the development of a diabetic complication. In some preferred embodiments, the diabetic complication is a microvascular complication, ischemia, or a neuronal cell or endothelial cell complication wherein, for example, neuronal or endothelial cells do not function normally and/or die. In other preferred embodiments, the diabetic complication is retinopathy which may, for example, comprise proliferative retinopathy or macular edema. In yet other preferred embodiments, the diabetic complication is nephropathy. In still other preferred embodiments, the diabetic complication is neuropathy which may include, for example, peripheral neuropathy, autonomic neuropathy, proximal neuropathy, or focal neuropathy.

In embodiments of the present disclosure, the TRPC6 inhibitor is a polypeptide, a protein, nucleic acid, or a small molecule. In certain embodiments, the TRPC6 inhibitor can be an antagonist that directly or indirectly decreases the amount or activity of a TRPC6 polypeptide. In certain embodiments, such an antagonist can be an antibody composition comprising an antibody that recognizes a TRPC6 polypeptide. The antibody may be, for example, a polyclonal antibody, monoclonal antibody, humanized antibody, single chain antibody, antibody fragment such as a Fab, or a bi-specific antibody.

In certain embodiments, the TRPC6 inhibitor is administered intravenously, intraarterially, intramuscularly, subcutaneously, or orally to the subject. In certain aspects, the TRPC6 inhibitor is administered on a daily, twice-daily, or three-times-daily basis. In some embodiments, the inhibitor is administered about every 4 hours, 6 hours, 8 hours, 12 hours, 24 hours, 48 hours, or 72 hours. In some aspects of the invention, the TRPC6 inhibitor is administered every other day, every third day, every fourth day, every fifth day, weekly, or monthly.

In one embodiment, a kit for treating hyperglycemia-induced platelet activation is provided, the kit comprising a TRPC6 inhibitor.

In further embodiments, the present invention provides kits for use with the disclosed methods and compositions regarding inhibition of platelet activation. Compositions comprising one or more inhibitors of TRPC6 may be provided in a kit. Such kits may be used to provide one or more such inhibitors in a ready to use and storable container.

It is contemplated that any method or composition described herein can be implemented with respect to any other method or composition described herein.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

The terms “effective” or “therapeutically effective,” as used in the specification and/or claims, means adequate to accomplish a desired, expected, or intended result.

Following long-standing patent law, the words “a” and “an,” when used in conjunction with the word “comprising” in the claims or specification, denotes one or more, unless specifically noted.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Mechanism for the activation of TRPC6 in human platelets.

FIGS. 2A-C. FIG. 2A shows super-imposed typical tracings for the [Ca²⁺]ᵢ of platelets pre-treated with specific antibody for TRPC6 or TRPC6 antibody blocked with the TRPC6 antigen (control) before and after the administration of collagen (reverse mode NCX agonist). FIG. 2B shows the collagen-induced change in [Ca²⁺]ᵢ for control platelets and platelets administered TRPC6 antibody. FIG. 2C shows super-imposed typical tracings for the collagen-induced platelet aggregation for platelets pre-treated with specific antibody for TRPC6 or TRPC6 antibody blocked with the TRPC6 antigen (control).

FIG. 3. Change in basal [Ca^{2+]}ᵢ for platelets incubated with TRPC6 antibody in 45 mM glucose for 4 hours compared to platelets incubated with TRPC6 antibody blocked with the TRPC6 antigen (control) in 45 mM glucose for 4 hours. ** p <0.005, n=6.

### DESCRIPTION OF ILLUSTRATIVE EMBODIMENTS

The present invention provides novel approaches to the treatment of hyperglycemia-induced microvascular complications by inhibiting TRPC6 activation and, thereby, inhibiting the NCX reverse mode and subsequent platelet activation.

A. HYPERGLYCEMIC DISORDERS AND MICROVASCULAR COMPLICATIONS

Plasma glucose levels are regulated by the action of insulin, a hormone that is produced and secreted by the pancreatic β-cells in response to nutrients and aids the body in converting sugars and other foods into energy. Diabetes mellitus, more commonly known as diabetes, encompasses a heterogeneous group of hyperglycemic disorders in which the body does not produce and/or properly use insulin. In a non-diabetic individual, insulin is produced in the pancreas by the β-cells present in the islets of Langerhans in response to increased glucose in the gut and/or blood. Insulin then acts in conjunction with the liver to control glucose metabolism in the body.

Although diabetes is typically thought of as a blood-sugar disease, diabetes may result in numerous life-threatening complications. While it is believed that diabetes is underreported as a cause of death, death certificates indicated that diabetes was the sixth leading cause of death in the United States in 2002. Complications of diabetes include microvascular disease, heart disease and stroke, high blood pressure, blindness, kidney disease, nervous system disease, amputation, dental disease, pregnancy complications, and increased susceptibility to other illnesses. Techniques currently employed to prevent diabetic complications include efforts to improve glucose control, blood pressure control, and blood lipid control and efforts to diagnose complications as early as possible. For example, improved glucose control reduces the risk of microvascular complications. Blood pressure control reduces the risk of heart disease and stroke. Improved control of blood lipids reduces the occurrence of cardiovascular complications. Early detection of eye disease and subsequent laser treatment reduces the occurrence of severe vision loss. Foot care programs reduce the incidence of amputation, and early detection and treatment of kidney disease, for example with ACE inhibitors and angiotensin receptor blockers, can aid in the maintenance of kidney function.

Several types of diabetes exist. Type 1 diabetes, previously called insulin dependent diabetes mellitus (IDDM) or juvenile-onset diabetes, is an auto-immune disease that destroys the insulin-producing pancreatic beta cells present in the islets of Langerhans, thereby destroying the body's ability to produce insulin. Type 1 diabetes most commonly occurs in children and young adults. Type 2 diabetes, previously called non-insulin dependent diabetes mellitus (NIDDM) or adult-onset diabetes, typically progresses from a condition in which the body does not use insulin properly, called insulin resistance, to a situation in which the pancreas can no longer produce insulin even though there is no significant loss in the number of pancreatic beta cells and these cells remain functional. Type 2 diabetes is most commonly diagnosed in older individuals and is associated with obesity, impaired glucose metabolism, physical inactivity, or a family history of diabetes or increased likelihood of developing diabetes due to race/ethnicity. Gestational diabetes is a third form of diabetes which occurs when a woman develops glucose intolerance during pregnancy. Treatment to normalize the mother's blood glucose level is required to prevent complications in the infant. Women who develop gestational diabetes are at an increased risk of developing diabetes in the five to ten years following the onset of gestational diabetes. Other types of diabetes may result from surgery, drugs, malnutrition, infection, and other illnesses.

While hyperglycemia is often symptomatic of diabetes, elevated blood glucose levels are not absolutely indicative of diabetes in the absence of other symptoms. For example, excessive white fat reserves in obese individuals may prevent the proper absorption and use of insulin. If non-diabetic hyperglycemia is chronic, similar complications as those observed in diabetic patients may result.

Microvascular complications induced by chronic hyperglycemia may include retinopathy, nephropathy, and neuropathy. Diabetic retinopathy is caused by diabetes-related changes in the vasculature of the retina. In some individuals with diabetic retinopathy, the blood vessels that supply the retina become swollen and may leak fluid. In others, diabetic retinopathy is marked by the abnormal new growth of blood vessels on the surface of the retina. Diabetic retinopathy is the leading cause of blindness in American adults.

In patients with diabetic retinopathy, blood vessel damage can promote vision loss in two ways. In proliferative diabetic retinopathy, abnormal blood vessels develop and leak blood into the center of the eye. Fluid can also leak into the macula and cause it to swell, a condition called macular edema. Vision is blurred in patients with either proliferative diabetic retinopathy or macular edema, and about half of diabetics who have proliferative retinopathy also have macular edema.

Diabetic patients are advised to keep their blood sugar under control in order to decrease their risk of developing retinopathy. Controlling blood pressure and blood cholesterol can also protect against vision loss in diabetic patients. Current treatments for proliferative retinopathy and macular edema typically involve laser eye surgery, which attempts to shrink the abnormal blood vessels. For patients who have a large amount of blood in the center of the eye, a vitrectomy may be required, in which the physician removes the vitreous gel that contains blood and replaces it with a salt solution. Macular edema is typically treated via a procedure called focal laser treatment, which aims to slow the leakage of fluid and decrease the amount of fluid in the retina.

Diabetic nephropathy (DN) is a microvascular complication of diabetes marked by a decreased ability to properly filter blood in the kidneys such that large blood proteins, such as albumin, pass into the urine and are lost. Diabetic nephropathy is the leading cause of kidney failure in the United States. It is characterized by albuminuria (excessive urine albumin excretion), with ultimate progression to end stage renal disease (ESRD), wherein the toxic substances in the blood build up to fatal levels. The risk of developing diabetic nephropathy varies based on ethnicity, gender, familial history of kidney disease or high blood pressure, time of onset of diabetes, ability to control blood sugar, high blood pressure, and smoking. The presence of diabetic nephropathy indicates that the individual may have blood vessel disease, and diabetic nephropathy is associated with an increased risk of heart attack, stroke and other circulatory malfunctions.

A number of approaches to controlling DN have been proposed. First and foremost, as with the treatment of diabetes generally, proper glycemic control helps limit DN. Second, Angiotensin-Converting Enzyme (ACE) and agiotensin II (AT II) receptor antagonists may be used to decrease proteinuria and slow the progression of kidney disease. In addition, diabetics who seek to decrease their risk of developing diabetic nephropathy are advised to control their blood pressure, control levels of blood lipids and cholesterol, stop smoking, and, under some circumstances, to reduce dietary protein intake.

Patients who develop diabetic neuropathy may experience pain, numbness, and weakness in the hands, arms, feet, and legs due to nerve damage. Neuropathies may affect any organ system in the body. There are four types of diabetic neuropathy: peripheral, autonomic, proximal, and focal.

Peripheral neuropathy results from nerve damage in the arms and legs and causes pain or numbness in toes, feet, legs, hands, and arms. Symptoms include numbness or insensitivity, sharp pain, cramps, extreme sensitivity, loss of balance, loss of coordination, and tingling, burning or prickling sensations. Foot injuries may occur and go unnoticed due to numbness, and therefore, such injuries may become severely infected and may even result in amputation of the foot.

Autonomic neuropathy promotes changes in autonomic functions including digestions, bowel function, bladder function, eye function, sexual response, perspiration, and blood pressure control. This type of neuropathy often affects the nerves that regulate the heart, blood pressure, and blood glucose levels. In some cases, autonomic neuropathy can prevent patients from being aware of a hypoglycemic state because the warning signs of hypoglycemia, including shakiness, no longer occur.

Patients with proximal neuropathy experience pain in the thighs, hips, buttocks, and weakness in the legs. It is more common in type II diabetics.

Focal neuropathy may affect any nerve in the body but commonly affects nerves associated with the eyes, facial muscles, ears, pelvis, lower back, thigh, and abdomen. In focal neuropathy, the sudden weakness of one nerve or a group of nerves occurs and causes muscle weakness or pain.

Diabetic patients are advised to keep their blood sugar under control in order to decrease their risk of developing diabetic neuropathy. In addition, diabetic neuropathy is more common in individuals who have high levels of blood fat, have high blood pressure, are overweight, or are over the age of 40.

Microvascular complications induced by hyperglycemia may also result from the formation of micro-aggregates that block small vessels denying the surrounding tissues of nutrients.

B. PLATELET ACTIVATION

Activated platelets adhere to each other and to endothelial cells in the walls of blood vessels forming a haemostatic plug in conjunction with fibrin. Abnormal platelet activation, therefore, results in the formation of platelet aggregates that can block small blood vessels. Calcium is an important second messenger for the activation of platelets. Elevations in [Ca²⁺]ᵢ influence almost all platelet responses including shape change, secretion, and thrombus formation. It has been reported that platelet Ca²⁺ homeostasis is abnormal in diabetes, and that there is a direct in vitro effect of elevated glucose concentration on platelet aggregation, Ca²⁺ homeostasis, and the Na⁺/Ca²⁺ (NCX) exchanger (Li et al., 2001). Therefore, it was deduced that the high [Ca²⁺]ᵢ induced by hyperglycemia contributes to the platelet hyper-responsiveness observed in diabetes.

In normal healthy platelets a resting [Ca²⁺]ᵢ of ˜100 nM is maintained by a balance between the leak of Ca²⁺ nto the platelet and the concurrent efflux of free Ca²⁺ across the plasma membrane of the platelet and accumulation in intracellular stores (Rink et al., 1982; Purdon et al., 1984). Ca²⁺ is moved out across the plasma membrane through the actions of the plasma membrane Ca²⁺-ATPase and the Na⁺/Ca²⁺ exchanger (NCX). Plasma membrane Ca²⁺-ATPases are membrane-inserted enzymes that use the energy of ATP hydrolysis to move Ca²⁺ against its gradient and across the membrane.

The NCX is a reversible carrier that can mediate the transport of Ca²⁺across the plasma membrane in both directions in exchange for Na⁺ (Rengasamy et al., 1987; Schaeffer and Blaustein, 1989). In most cells and situations the role of the NCX is to remove Ca²⁺ from the cell (forward mode); however, under some conditions the exchanger can mediate the net influx of Ca²⁺ (reverse mode). In the resting state, the NCX removes Ca²⁺ from the platelet cytosol. Internally, Ca²⁺ is transported into the dense tubular system by the sarco/endoplasmic reticulum Ca²⁺-ATPases 2b and 3 (Papp et al., 1991; Wuytack et al., 1994).

Because voltage-gated calcium channels are not present in platelets (Pipili et al., 1985; Sage and Rink, 1986; Doyle and Ruegg, 1985; Motulsky et al., 1983), either a receptor-operated calcium channel or a reverse mode NCX could contribute to the influx of Ca²⁺ (Rengasamy et al., 1987; Schaeffer and Blaustein, 1989; Mahaut-Smith et al., 1990). An initial influx of Na⁺ is needed for the reversal of NCX. Influx of Na³⁰ has been shown upon activation of platelets by thrombin, ADP, polylysine, and collagen (Greenberg-Sepersky and Simons, 1984; Feinberg et al., 1977; Sandler et al., 1980; Roberts et al., 2004). Matsuoka and Hilgemann (1992) reported that at a [Ca²⁺]ᵢ of 100 nM and a membrane potential of -60 mV (platelet membrane potential at rest and in response to collagen (Maclntyre and Rink, 1982), a small change in [Na³⁰ ]ᵢ can result in the reversal of the NCX. Na³⁰ /H⁺ exchangers (NHEs) could also contribute to the increase in [Na³⁰ ]ᵢ and subsequent NCX reversal.

The reverse mode of the NCX has been reported in diabetes and other disease states. For example, in central nervous system anoxia/ischemia, most of the Ca²⁺ nflux in the white matter is mediated by a reverse mode of the NCX (Stys and Steffensen, 1996). Even in certain normal physiological conditions, the NCX has been described as mediating Ca²⁺ entry, e.g., in cardiac cells (Leblanc and Hume, 1990) and lymphocytes (Balasubramanyam et al., 1994).

C. TRANSIENT RECEPTOR POTENTIAL (TRP) ION CHANNELS

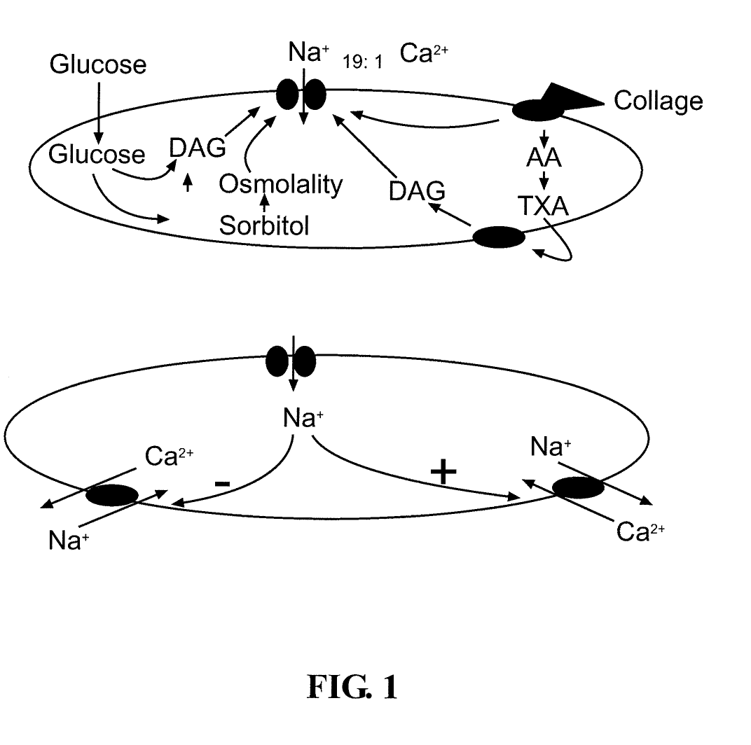

The inventors have shown that TRPC6 is involved in platelet activation. As illustrated in FIG. 1, hyperglycemia results in the activation of TRPC6, via (a) the generation of diacylglyceride (DAG), and/or (b) an increase in platelet osmolarity. Activation of TRPC6 results in the influx of Na³⁰ . This leads to an increase in the [Na³⁰ ]ᵢ, which disrupts the Na³⁰ gradient and results in the inhibition of the forward mode of the Na³⁰ /Ca²⁺ exchanger (NCX) initially and subsequent reversal of the NCX. Reversal of the NCX leads to an increase in cytosolic Ca²⁺ ([Ca²⁺]ᵢ), which generates hyper-responsive platelets and the formation of micro-aggregates.

The Transient Receptor Potential (TRP) superfamily is one of the largest ion channel families. There are about 28 genes encoding the TRP ion channel subunits in mammals. The mammalian TRP superfamily comprises six subfamilies known as the TRPC (canonical), TRPV (vanilloid), TRPM (melastatin), TRPML (mucolipins), TRPP (polycystin), and the TRPA (ANKTM1) ion channels (Moran et al., 2004; Clapham et al., 2003; Clapham, 2003; Padinjat and Andrews, 2004). The TRPC subfamily consists of seven proteins named TRPC1 to 7, which can be further divided into four subgroups based on their sequence homology and functional similarities: (1) the TRPC1; (2) TRPC4 and TRPC5; (3) TRPC3, TRPC6, TRPC7; and (4) TRPC2 (Clapham et al. 2003; Huang, 2004). TRPC6 can form heterotetramers with TRPC3 and TRPC7. TRPC6 is primarily expressed in brain, lung and muscle, with high levels of expression also found in human platelets.

D. TRPC INHIBITORS

The inventors demonstrated that by inhibiting TRPC6, NCX reversal and the subsequent increase in platelet [Ca²⁺]ᵢ can be inhibited. Thus, the use of inhibitors that target TRPC6 provide a novel approach to treating or preventing hyperglycemia-induced platelet activation.

The TRPC6 inhibitor(s) used to prevent or treat platelet activation according to the present invention can be a polypeptide, a protein, a nucleic acid, or a small molecule. The TRPC6 inhibitor(s) can directly or indirectly decrease the amount or activity of a TRPC6 polypeptide. In certain embodiments, the TRPC6 inhibitor can be an antagonist of TRPC6. The antagonist may be an antibody composition comprising an antibody that recognizes a TRPC6 polypeptide. The antibody may be, for example, a polyclonal antibody, monoclonal antibody, humanized antibody, single chain antibody, antibody fragment such as a Fab, or a bi-specific antibody.

In certain embodiments, isolated antibodies to TRPCs of the present disclosure, such as TRPC6, are contemplated as useful for purification, diagnostic and therapeutic applications. Monoclonal antibodies (MAbs) are recognized to have certain advantages, e.g., reproducibility and large scale production, and their use is generally preferred. MAbs may be readily prepared through use of well known techniques, such as those exemplified in U.S. Pat. No. 4,196,265, incorporated by reference.

E. PHARMACEUTICAL COMPOSITIONS

Where clinical applications are contemplated, it will be necessary to prepare pharmaceutical compositions of the present invention in a form appropriate for administration to a subject. The compositions will generally be prepared as essentially free of impurities that could be harmful to humans or animals. The preparation of a pharmaceutical composition including a TRPC6 inhibitor will be known to those of skill in the art in light of the present disclosure, as exemplified by Remington's Pharmaceutical Sciences, 18th Ed. Mack Printing Company, 1990, incorporated by reference. Moreover, for animal (e.g., human) administration, it will be understood that pharmaceutical preparations should meet sterility, pyrogenicity, general safety and purity standards as required by FDA Office of Biological Standards. Therefore, one will generally desire to employ appropriate salts and buffers to render stable formulations suitable for introduction into a patient. Aqueous compositions of the present invention comprise an effective amount of inhibitor dispersed in a pharmaceutically or pharmacologically acceptable carrier.

The phrases “pharmaceutically acceptable” and “pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (Remington's, 1990). The use of such carriers for pharmaceutically active substances is well know in the art. Except insofar as any conventional carrier is incompatible with the inhibitors of the present invention, its use in therapeutic compositions is contemplated. Supplementary active ingredients also can be incorporated into the compositions.

Under ordinary conditions of storage and use, pharmaceutical preparations may further contain a preservative to prevent growth of microorganisms. Intravenous vehicles include fluid and nutrient replenishers. Preservatives include antimicrobial agents, antioxidants, chelating agents and inert gases. The pH and exact concentration of the various components in the pharmaceutical are adjusted according to well-known parameters.

An effective amount of a therapeutic composition is determined based on the intended goal. “Effective amounts” are those amounts effective to produce beneficial results in the recipient animal or patient. Such amounts may be initially determined by reviewing the published literature, by conducting in vitro tests or by conducting metabolic studies in healthy experimental animals. Before use in a clinical setting, it may be beneficial to conduct confirmatory studies in an animal model, preferably a widely accepted animal model of the particular disease to be treated. Preferred animal models for use in certain embodiments are rodent models, which are preferred because they are economical to use and, particularly, because the results gained are widely accepted as predictive of clinical value. The term “unit dose” refers to a physically discrete unit suitable for use in a subject, each unit containing a predetermined quantity of the composition calculated to produce the desired response in association with its administration, i.e., the appropriate route and treatment regimen. The quantity to be administered, both according to number of treatments and unit dose, depends on the subject to be treated, the state of the subject, and the protection desired. Precise amounts of the therapeutic composition also depend on the judgment of the practitioner. The actual dosage amount of a composition of the present invention administered to a patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

The composition may comprise various antioxidants to retard oxidation of one or more components. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The compositions of the present invention may comprise different types of carriers depending on whether it is to be administered in solid, liquid or aerosol form, and whether it needs to be sterile for such routes of administration as injection.

The compositions may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments, the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments, the compositions are prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir may comprise, for example, at least one active agent, a sweetening agent, a preservative, a flavoring agent, a dye, a preservative, or combinations thereof.

In certain embodiments, an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The pharmaceutical compositions of the present invention may be administered intravenously, intradermally, intraarterially, intraperitoneally, intralesionally, intracranially, intraarticularly, intraprostaticaly, intrapleurally, intratracheally, intranasally, intravitreally, intravaginally, intrauterinely, intrarectally, intrathecally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (Remington's, 1990). Oral administration of the composition includes oral, buccal, enteral or intragastric administration.

F. KITS

In further embodiments, the present invention concerns kits for use with the disclosed methods regarding inhibition of platelet activation. Compositions comprising one or more inhibitors of TRPC6 may be provided in a kit. Such kits may be used to provide one or more such inhibitors in a ready to use and storable container.

The container of the kits can generally include at least one vial, test tube, flask, bottle, syringe and/or other container, into which at least one inhibitor composition, such as an antibody, may be placed, and/or preferably, suitably aliquoted. The kits of the present invention may include a means for containing inhibitor components, inhibitors or any other reagent containers in close confinement for commercial sale. Such containers may include injection and/or blow molded plastic containers into which the desired vials are retained.

G. COMBINATION THERAPIES

In accordance with the present invention, it may prove advantageous to combine the methods disclosed herein with adjunct therapies or procedures for treating diabetes or microvascular complications to enhance the overall therapeutic effect. One such combination therapy may comprise combining administration of a TRPC6 inhibitor with aspirin therapy. The compositions of the present invention may be administered at the same time as the other therapy or it may precede or follow the other therapy by intervals ranging from minutes to weeks. It is contemplated that one may administer both modalities within about 12-24 h of each other and, more preferably, within about 6-12 h of each other. In some situations, it may be desirable to extend the time period for treatment significantly, where several days (2, 3, 4, 5, 6 or 7) to several weeks (1, 2, 3, 4, 5, 6, 7 or 8) lapse between the respective administrations.

Various combinations may be employed where a compositions including an inhibitor of platelet activation or aggregation, such as a TRPC6 inhibitor is “A” and the secondary agent, is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

H. SCREENING METHODS

The present invention also contemplates the identification of TRPC6 inhibitors and screening of putative TRPC6 inhibitors for their effectiveness in preventing platelet activation and/or aggregation. In particular aspects, the screen may be designed to identify compounds which inhibit platelet aggregation in vitro or in vivo or promote reverse function of NCX.

Models of Type I diabetes include streptozotocin-treated animals and mice with a mutation in the Ins2 gene (Akita mice), which are commercially available (Jackson Labs). Other models of diabetes may also prove useful. B6.HRS(BKS)-Cpefat/+(Jackson Labs) is a C57BL/6J congenic strain carrying the fat spontaneous mutation. B6.HRS(BKS)-Cpefat/+mice have been backcrossed to C57BL/6J for 10 generations (N10). Homozygous Cpefat mice develop a diabetic phenotype characterized by hyperglycemia and insulin resistance. C57BL/6J mutant mice also develop obesity at an earlier age than BKS.HRS-Cpefat/J mice (Jackson Labs), with the females becoming heavier than males. Obesity develops later than in obese (B6.V-Lepob; Jackson Labs) and diabetes (BKS.Cg-m +/+Leprdb; Jackson Labs) mutant mice. Cpefat mice actually weigh less than wildtype controls prior to weaning age (Weide & Lacy, 1991; Naggert et al., 1995). C57BL/6-Ins2Akita (Jackson Labs) is another diabetes model associated with proinsulin processing defects.

One important aspect of the present invention concerns assays for screening for potential TRPC6 inhibitors that can be used in therapeutic applications. A TRPC6 inhibitor refers to a compound that is able to reduce effective TRPC6 amount or functional activity. These assays may comprise random screening of large libraries of candidate substances; alternatively, the assays may be used to focus on particular classes of compounds selected with an eye towards structural attributes that are believed to make them more likely to modulate TRPC6. To identify an inhibitor of TRPC6, one generally will determine the activity of TRPC6 in the presence and absence of a candidate substance, wherein an inhibitor of TRPC6 is defined as any substance that alters the amount or activity of TRPC6.

Assays may be conducted in cell free systems, in isolated cells, or in organisms including transgenic animals. It will, of course, be understood that all the screening methods of the present invention are useful in themselves notwithstanding the fact that effective candidates may not be found. The invention provides methods for screening for such candidates, not solely methods of finding them.

As used herein the term “candidate substance” refers to any molecule that may potentially inhibit the effective level of TRPC6 activity or expression. A TRPC6 inhibitor refers to a substance that decreases or reduces the effective level of TRPC6 activity or expression. It is contemplated that the term inhibitor is relative to conditions when the inhibitor is not present.

Candidate substances can include fragments or parts of naturally-occurring compounds or may be only found as active combinations of known compounds which are otherwise inactive. In one embodiment, the candidate substances are small molecules. In yet other embodiments, candidate substances may be synthetic or natural peptides. Examples of small molecules that may be screened include, but are not limited to, small organic molecules, peptides or fragments thereof, peptide-like molecules, nucleic acids, polypeptides, peptidomimetics, carbohydrates, lipids or other organic (carbon-containing) or inorganic molecules. Many pharmaceutical companies have extensive libraries of chemical and/or biological mixtures, often fungal, bacterial, or algal extracts, which can be screened with any of the assays of the invention to identify compounds that modulate TRPC6 activity.

Alternatively, it is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds.

Candidate substances identified may then be tested in biochemical or biological assays to further identify TRPC6 modulators. Functional assays can also be employed to characterize candidate substances. Moreover, one or more assays may be employed for quality control evaluations once a particular candidate substance is determined to be a TRPC6 inhibitor for pharmaceutical formulation.

The present invention also provides methods for developing drugs that inhibit TRPC6 activity, which may be used to prevent or treat hyperglycemia-induced complications. One such method involves the prediction of the three dimensional structure of TRPC6 or a substrate thereof using molecular modeling and computer stimulations. The resulting structure may then be used in docking studies to identify potential small molecule inhibitors that bind in the enzyme's active site with favorable binding energies.

Rational drug design is therefore used to produce structural analogs of substrates for TRPC6. By creating such analogs, it is possible to fashion drugs having biological activity. In one approach, one would generate a three-dimensional structure for the TRPC6 targets of the invention or a fragment thereof. This could be accomplished by X-ray crystallography, computer modeling or by a combination of both approaches.

It also is possible to use antibodies to ascertain the structure of a target compound modulator. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators. Another suitable compound includes antibodies (including single chain antibodies). Such compounds are described in greater detail elsewhere in this document.

I. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Platelet Isolation: Venous blood was drawn from healthy volunteers, who denied taking aspirin for at least 14 days prior to participation, into EDTA containing vacutainer tubes. Platelet rich plasma was isolated from blood samples by centrifugation at 600 rpm for 15 minutes. Platelets were isolated from platelet rich plasma by centrifugation at 2000 rpm for 15 minutes. Platelet samples were re-suspended in 500 μl of platelet poor plasma and incubated at 37° C. for 1 hour with the calcium sensitive fluorescent dye fura-2-AM (10 μM) (Molecular Probes, Eugene, Oreg.).

Following incubation to load the dye, the platelets were separated from the plasma and extracellular dye by gel filtration using a Sepharose CL-2B column. The platelets were eluted in a Ca²⁺ free HEPES buffer containing (in mM): 140 NaCl, 4.9 KCl, 1.2 MgCl₂, 1.4 KH₂PO₄, 5.5 glucose, and 20 HEPES (pH 7.4), counted in a Coulter counter and adjusted to 2×10⁸ platelets/ml.

Fluorescence and Aggregation Measurement: Changes in fluorescence and aggregation were simultaneously measured at 37° C. in a Jasco Inc., Model CAF-110 Ion Analyzer (Easton, Md.). The excitation wavelength for [Ca²⁺]ᵢ measurement was 340 nm and 380 nm and the emission wavelength was 500 nm. [Ca²⁺]ᵢ was calculated according to previously published formula (Grynkiewicz et al., 1985; Li et al., 2001; Roberts et al., 2004). Aggregation was measured as a change in optical density using a near-infrared LED 950 nm light source.

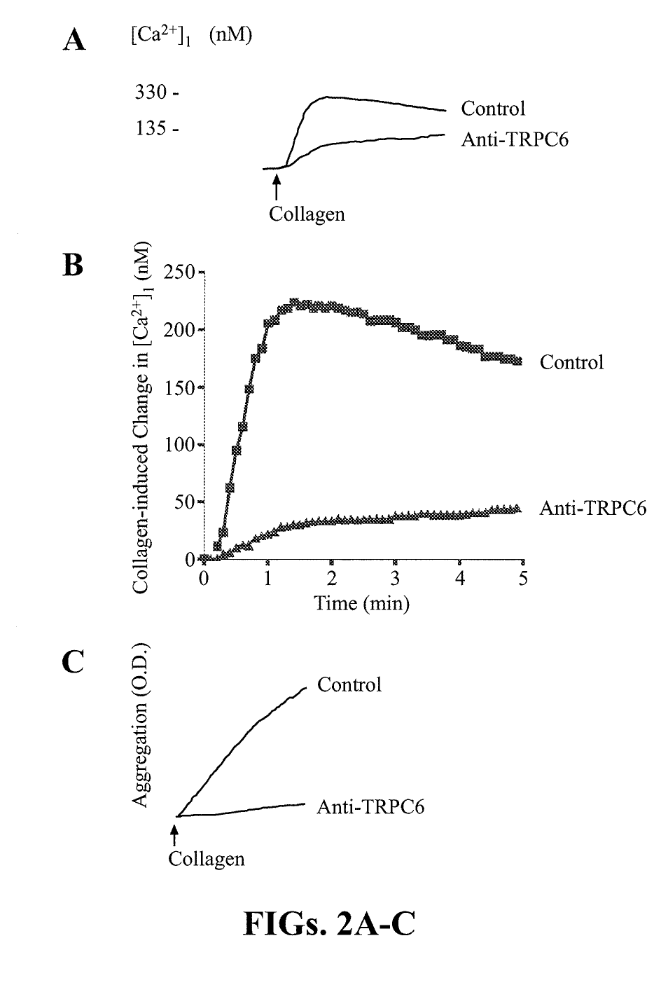

Role of TRPC6 in agonist induced Na³⁰ /Ca²⁺ exchanger reversal: Collagen activation of platelets is similar to that seen with hyperglycemia. Collagen acts on TRPC6 to increase cytosolic Na³⁰ , causing the NCX to reverse thereby increasing cytosolic Ca²⁺, which leads to platelet aggregation. Aliquots of the platelets were incubated for 30 minutes at room temperature with either anti-TRPC6 polyclonal antibody (Alomone Labs, Jerusalem, Israel) or anti-TRPC6 polyclonal antibody and TRPC6 antigen, and then placed in the ion analyzer to simultaneously measure changes in fluorescence and aggregation. Samples were incubated with 1 mM Ca²⁺ at 37° C. for 3 minutes prior to the administration of 10 μg/mL collagen. The anti-TRPC6 antibody prevented the collagen-induced NCX reversal and subsequent increase in cytosolic Ca²⁺ (FIGS. 2A and 2B). Reducing the collagen-induced increase in cytosolic Ca²⁺ translated into a reduction in the platelet aggregation (FIG. 2C).

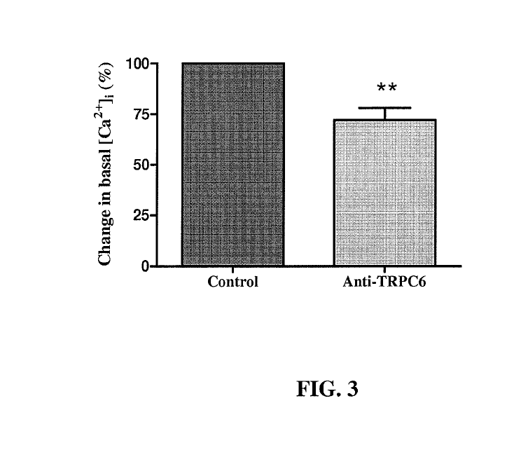

Role of TRPC6 in hyperglycemia induced change in basal calcium: Fura-2 loaded platelets were suspended in autologous plasma supplemented with glucose to a final concentration of 45 mM. Samples were then incubated with either anti-TRPC6 polyclonal antibody or anti-TRPC6 polyclonal antibody and TRPC6 antigen for 4 hours at 37° C. Platelets were isolated via centrifugation at 2000 rpm for 10 minutes and re-suspended in Ca²⁺ free HEPES buffer. Samples were incubated with 1mM Ca²⁺ at 37° C. for 3 minutes while changes in fluorescence were measured. As shown in FIG. 3, the change in basal [Ca²⁺]ᵢ for platelets incubated with TRPC6 antibody in 45 mM glucose for 4 hours was significantly less compared to platelets incubated with TRPC6 antibody blocked with the TRPC6 antigen (control) in 45 mM glucose for 4 hours (p <0.005, n=6).

Chemicals: Fura-2-AM was purchased from Molecular Probes (Eugene, Oreg.), and dissolved in dimethyl sulfoxide (DMSO). Anti-Transient receptor potential like channel 6 (TRPC6) polyclonal antibody was purchased from Alomone Labs (Jerusalem, Israel). Collagen was obtained from Nycomed Arzneimittel (Munich, Germany). Sepharose 2B-CL was obtained from Pharmacia Biotechnology. All other chemicals were purchased from Sigma.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,196,265
Balasubramanyam et al., J. Clin. Invest., 94:2002-2008, 1994.
Clapham et al., Pharmacol. Rev., 55:591, 2003.
Clapham, Nature, 426:517, 2003.
Collwell et al., Diabetes Care, 4:121-133, 1981.
Colwell et al., Am. J. Med., 75:67-80, 1983.
Dobbie et al., Arch. Ophthalmol., 91:107-109, 1974.
Doyle and Ruegg, Biochem. Biophys. Res. Commun., 127:161-167, 1985.
Feinberg et al., Biochim. Biophys. Acta, 470:317-324, 1977.
Greenberg-Sepersky and Simons, J. Biol. Chem., 259:1502-1508, 1984.
Grynkiewicz et al., J. Biol. Chem., 260:3440-3450, 1985.
Honour et al., Br. J. Exp. Pathol., 57:1-10, 1976.
Huang, J. Am. Soc. Nephrol., 15:1690, 2004.
Ihibashi et al., Diabetes, 30:601-606, 1981.
Leblanc and Hume, Science, 248:372-376, 1990.
Li et al., Amer. J. Physiology, 280:H1480-14889, 2001.
Maclntyre and Rink, Thromb. Haemostasis, 47:22-26, 1982.
Mahaut-Smith et al., J. Biol. Chem., 265:10479-10483, 1990.
Matsuoka and Hilgemann, J. Gen. Physiol., 100:963-1001, 1992.
Moran et al., Current Opin. Neurobiol., 14:362, 2004.
Morrish et al., Diabetologia, 33:538-541, 1990.
Motulsky et al., Circ. Res., 52:226-231, 1983.
Naggert et al., Nat. Genet., 10(2):135-142, 1995.
Padinjat and Andrews, J. Cell. Sci., 117:5707, 2004.
Papp et al., J. Biol. Chem., 266:14593-14596, 1991.
Patrono et al., Diabetes Metab., 9:177-188, 1993.
Pipili, Thromb. Haemostasis, 54:645-649, 1985.
Purdon et al., Biochim. Biophys. Acta, 800:178-187, 1984.
Pyorala et al., Diabetes Metab. Rev., 3:463-524, 1987.
Remington's Pharmaceutical Sciences, 18th Ed. Mack Printing Company, pp. 1289-1329, 1990.
Rengasamy et al., Thromb. Haemostasis, 57:337-340, 1987.
Rink et al., FEBS Lett., 148, 21-26, 1992.
Roberts etal., J. Biol. Chem., 279:19421-19430, 2004.
Sage and Rink, Biochem. Biophys. Res. Commun., 136:1124-1129, 1986.
Sandler et al., Biochim. Biophys. Acta, 600:448-455, 1980.
Schaeffer and Blaustein, Cell Calcium, 10:101-113, 1989.
Stys and Steffensen, Ann. NY Acad. Sci., 779:366-378, 1996.
Weide & Lacy, Diabetes, 40(9):1157-1162, 1991.
Wuytack et al., J. Biol. Chem., 269:1410-1416, 1994.

What is claimed is:

1. A method of inhibiting hyperglycemia-induced platelet activation comprising contacting platelets with an effective amount of an antibody that binds TRPC6, wherein binding of the antibody to the TRP6 of the platelets inhibits hyperglycemia-induced platelet activation.

2. The method of claim 1, wherein inhibiting platelet activation comprises inhibiting platelet aggregation.

3. The method of claim 1, wherein the antibody is a monoclonal antibody.

4. The method of claim 1, wherein the antibody is a polyclonal antibody.

5. The method of claim 1, wherein the antibody is an antibody fragment.

6. The method of claim 1, wherein the platelets are contacted in vitro.

7. The method of claim 1, wherein the platelets are contacted in vivo.

8. A method of treating a diabetic complication in a subject having diabetes comprising administering to the subject an effective amount of an antibody that binds TRPC6, wherein the diabetic complication is treated.

9. The method of claim 8, wherein the antibody is administered prior to the development of the diabetic complication.

10. The method of claim 8, wherein the antibody is administered after the development of the diabetic complication.

11. The method of claim 8, wherein the antibody is administered intravenously.

12. The method of claim 8, wherein the diabetic complication is a microvascular complication.

13. The method of claim 8, wherein the diabetic complication is ischemia.

14. The method of claim 8, wherein the diabetic complication is a retinopathy.

15. The method of claim 8, wherein the diabetic complication is a nephropathy.

16. The method of claim 8, wherein the diabetic complication is a neuropathy.

17. The method of claim 8, wherein the diabetic complication is an endothelial cell complication.

18. The method of claim 8, wherein the antibody is a monoclonal antibody.

19. The method of claim 8, wherein the antibody is a polyclonal antibody.

20. The method of claim 8, wherein the antibody is an antibody fragment.

21. A method of treating a microvascular complication in a hyperglycemic subject comprising administering to the subject an effective amount of an antibody that binds TRPC6, wherein the microvascular complication is treated.

22. The method of claim 21, further comprising assessing the blood glucose level of the subject.

23. The method of claim 21, wherein the hyperglycemic subject has diabetes.

24. The method of claim 21, wherein the microvascular complication is a retinopathy, neuropathy, or nephropathy.

25. The method of claim 21, wherein the microvascular complication is ischemia.
